(19) United States
(12) Patent Application Publication
(10) Pub. No.: US 2020/0129509 A1
(43) Pub. Date: Apr. 30, 2020

(54) DOSING REGIMEN

(71) Applicant: Cyclacel Limited, London (GB)

(72) Inventor: Judy H. CHIAO, Berkeley Heights, NJ (US)

(21) Appl. No.: 16/171,719

(22) Filed: Oct. 26, 2018

(51) Int. Cl. A61K 31/506; A61K 31/7068; A61P 35/02; A61K 9/00

(52) U.S. Cl. CPC A61K 31/506; A61K 31/7068; A61K 9/0053; A61K 9/0019; A61P 35/02

(57) ABSTRACT

The invention relates to a method of treating AML in a subject having a white blood cell (WBC) count of less than about 10,000 cells/microliter, and/or a cytogenetic risk classification according to the US Southwest Oncology Group (SWOG) that is not unfavourable, and/or the subject falls within a classification selected from antecedent myelodysplastic syndrome (MDS), antecedent myeloproliferative neoplasm (MPN), and antecedent myelodysplastic/myeloproliferative neoplasm (MDS/MPN), wherein the method comprises (i) a first treatment cycle comprising administering decitabine for 5 to 10 consecutive days followed by a rest period of from 3 to 5 weeks, or until treatment-related toxicities are resolved, whichever is longer; and (ii) a second treatment cycle comprising administering sapacitabine, or a metabolite thereof, for 3 consecutive days per week, for 2 weeks followed by a rest period of from 2 to 4 weeks, or until treatment-related toxicities are resolved, whichever is longer.

### FIELD OF THE INVENTION

The present invention relates to a dosing regimen suitable for the treatment of acute myeloid leukemia (AML) in a defined subgroup of patients. In particular, the invention relates to a dosing regimen comprising the administration of decitabine in combination with sapacitabine, or a metabolite thereof.

### BACKGROUND TO THE INVENTION

DNA methyltransferases are a family of enzymes that promote the covalent addition of a methyl group to a specific nucleotide base in a molecule of DNA. All the known DNA methyltransferases use S-adenosyl methionine (SAM) as the methyl donor. Four active DNA methyltransferases have been identified in mammals. They are named DNMT1, DNMT2, DNMT3A and DNMT3B.

DNMT1 is the most abundant DNA methyltransferase in mammalian cells and considered to be the key maintenance methyltransferase in mammals. It predominantly methylates hemimethylated CpG di-nucleotides in the mammalian genome and is responsible for maintaining methylation patterns established in development. The enzyme is about 1620 amino acids long, the first 1100 amino acids constituting the regulatory domain, and the remaining residues constituting the catalytic domain. These are joined by Gly-Lys repeats. Both domains are required for the catalytic function of DNMT1. DNMT3 is a family of DNA methyltransferases that can methylate hemimethylated and unmethylated CpG at the same rate. The architecture of DNMT3 enzymes is similar to DNMT1 with a regulatory region attached to a catalytic domain.

Recent work has revealed how DNA methylation and chromatin structure are linked at the molecular level and how methylation anomalies play a direct causal role in tumorigenesis and genetic disease. Much new information has also come to light regarding DNA methyltransferases, in terms of their role in mammalian development and the types of proteins they are known to interact with. Rather than enzymes that act in isolation to copy methylation patterns after replication, the types of interactions discovered thus far indicate that DNA methyltransferases may be components of larger complexes actively involved in transcriptional control and chromatin structure modulation. These findings should enhance the understanding of the myriad roles of DNA methylation in disease, as well as leading to novel therapies for preventing or repairing these defects.

Small molecule DNA methyltransferase inhibitors are well documented in the art and include, for example, decitabine, azacitidine, zebularine, procainamide, procaine, hydralazine, (−)-epigallocatechin-3-gallate (EGCG) and RG108.

It is well established in the art that active pharmaceutical agents can often be given in combination in order to optimise the treatment regime.

Qin T et al (2007, 13, Clin. Cancer Res. 4225-4232) disclose the effect of combinations of cytarabine and decitabine in various human leukemic cell lines. Likewise, Kong X B et al (1991, Molecular Pharmacol. 39, 250-257) suggest that 5-azacitidine causes upregulation of deoxycytidine kinase (dCK) in a cell line that is resistant to cytarabine, resulting in a decrease in the IC₅₀ value for cytarabine from 12.5 to 0.55 μM.

Combinations of DNA methyltransferase inhibitors and 1-(2-C-cyano-2-deoxy-β-D-arabino-pentofuranosyl)-N4-palmitoyl cytosine (also known as “CYC682” or sapacitabine), or a metabolite thereof, are described in WO 2009/150405 (Cyclacel Limited). Pharmaceutical compositions comprising such combinations, and their use in treating various proliferative disorders are also described in WO 2009/150405.

WO 2012/140436 (Cyclacel Limited) describes an alternating dosing regimen for the treatment of acute myeloid leukemia (AML) using sapacitabine and decitabine. Ravandi et al (Abstract 2630; December 2012; American Society of Hematology) describes pooled patient data for a Phase 1/2 trial in AML patients of greater than 70 years of age, treated in accordance with a dosing regimen comprising administering decitabine at a rate of 20 mg/m² for 5 days a week of a 4 week cycle (odd cycles), and administering sapacitabine at a rate of 300 mg b.i.d. for 3 days a week for 2 weeks of 4 week cycle (even cycles). The results of this study indicated that the sequential combination of decitabine and sapacitabine appears to be safe and effective.

The present invention seeks to provide a dosing regimen for decitabine and sapacitabine that is suitable for the treatment of AML in a new subgroup of patients.

### STATEMENT OF INVENTION

A first aspect of the invention relates to a method of treating AML in a subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

said method comprising administering to the subject a therapeutically effective amount of (i) sapacitabine, or a metabolite thereof; and (ii) decitabine; in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

Surprisingly, the applicant has found that patients with a WBC count of <10,000 cells/microliter experience better efficacy when treated with sapacitabine/decitabine than with decitabine alone, and compared to patients treated with sapacitabine/decitabine and having a WBC count of 10,000 cells/microliter. The applicant has also found that the cytogenetic classification of “not unfavourable” further selects for sensitive patients, as does antecedent MPN/MDS status.

A second aspect of the invention relates to a method of treating AML in a subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

said method comprising administering to a subject a therapeutically effective amount of (i) sapacitabine; and (ii) decitabine; in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

A third aspect of the invention relates to a method of treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

said method comprising administering to a subject a therapeutically effective amount of (i) sapacitabine; and (ii) decitabine; in accordance with a dosing regimen comprising at least one first treatment cycle and at least one second treatment cycle,
wherein said first treatment cycle comprises administering decitabine intravenously in a dose of about 20 mg/m² per day for 5 or 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer.

A fourth aspect of the invention relates to (i) sapacitabine, or a metabolite thereof; and (ii) decitabine; for use in treating AML in a subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

wherein the sapacitabine, or a metabolite thereof, and the decitabine are administered in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

A fifth aspect of the invention relates to (i) sapacitabine, or a metabolite thereof; and (ii) decitabine; for use in treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

and wherein the sapacitabine, or metabolite thereof, and decitabine, are administered in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer.

A sixth aspect of the invention relates to use of (i) sapacitabine, or a metabolite thereof; and (ii) decitabine; in the preparation of a medicament for treating AML in a subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

and wherein the sapacitabine, or a metabolite thereof, and the decitabine are administered in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

A seventh aspect of the invention relates to a use of (i) sapacitabine, or a metabolite thereof; and (ii) decitabine; in the preparation of a medicament for treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

and wherein the sapacitabine, or metabolite thereof, and decitabine, are administered in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer.

An eighth aspect of the invention relates to a kit of parts comprising:

(i) sapacitabine, or a metabolite thereof;

(ii) decitabine; and

(ii) instructions for administering sapacitabine, or a metabolite thereof, and decitabine to a subject, in accordance with a dosing regimen comprising at least one first treatment cycle and at least one second treatment cycle, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

A ninth aspect of the invention relates to a kit of parts comprising:

(i) sapacitabine, or a metabolite thereof;

(ii) decitabine; and

(iii) instructions for administering sapacitabine, or a metabolite thereof, and decitabine to a subject in accordance with a dosing regimen comprising at least one first treatment cycle and at least one second treatment cycle, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein said first treatment cycle comprises administering decitabine intravenously in a dose of about 20 mg/m² for 5 to 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer.

### DETAILED DESCRIPTION

The presently claimed dosing regimen is well tolerated and gives rise to excellent response rates, good overall survival rates and absence of overlapping or cumulative toxicities.

1-(2-C-cyano-2-deoxy-β-D-arabino-pentofuranosyl)-N⁴-palmitoyl cytosine (I), also known as 2′-cyano-2′-deoxy-N⁴-palmitoyl-1-β-D-arabinofuranosylcytosine (Hanaoka, K., et al, Int. J. Cancer, 1999: 82:226-236; Donehower R, et al, Proc Am Soc Clin Oncol, 2000: abstract 764; Burch, P A, et al, Proc Am Soc Clin Oncol, 2001: abstract 364), is an orally administered novel 2′-deoxycytidine antimetabolite prodrug of the nucleoside CNDAC, 1-(2-C-Cyano-2-deoxy-β-D-arabino-pentafuranosyl)-cytosine or 2′-C-cyano-2′-deoxy-1-β-D-arabino-pentofuranosyl cytosine.

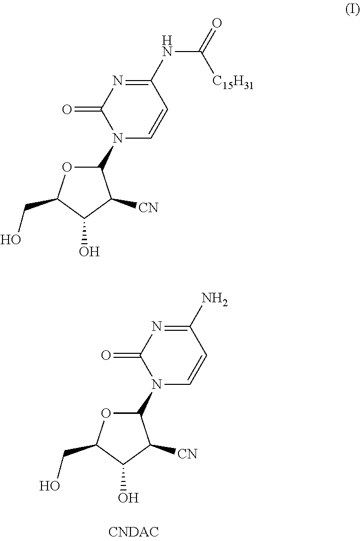

Sapacitabine

1-(2-C-cyano-2-deoxy-β-D-arabino-pentofuranosyl)-N⁴-palmitoyl cytosine (I) (also known as “CYC682” or sapacitabine) has a unique mode of action over other nucleoside metabolites such as gemcitabine in that it has a spontaneous DNA strand breaking action, resulting in potent anti-tumour activity in a variety of cell lines, xenografts and metastatic cancer models.

1-(2-C-cyano-2-deoxy-β-D-arabino-pentofuranosyl)-N⁴-palmitoyl cytosine (I) has been the focus of a number of studies in view of its oral bioavailability and its improved activity over gemcitabine (the leading marketed nucleoside analogue) and 5-FU (a widely-used antimetabolite drug) based on preclinical data in solid tumours. Recently, investigators reported that (I) exhibited strong anticancer activity in a model of colon cancer. In the same model, (I) was found to be superior to either gemcitabine or 5-FU in terms of increasing survival and also preventing the spread of colon cancer metastases to the liver (Wu M, et al, Cancer Research, 2003: 63:2477-2482). To date, phase I data from patients with a variety of cancers suggest that (I) is well tolerated in humans, with myelosuppression as the dose-limiting toxicity.

The DNA methyltransferase inhibitor used in the dosing regimen of the present invention is decitabine. Decitabine or 5-aza-2′-deoxycytidine (trade name Dacogen) is the compound 4-amino-1-(2-deoxy-β-D-erythro-pentofuranosyl)-1,3,5-triazin-2(1H)-one, the structure of which is shown below.

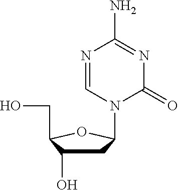

Decitabine is indicated for the treatment of myelodysplastic syndromes (MDS) including previously treated and untreated, de novo and secondary MDS of all French-American-British subtypes (refractory anemia, refractory anemia with ringed sideroblasts, refractory anemia with excess blasts, refractory anemia with excess blasts in transformation, and chronic myelomonocytic leukemia) and Intermediate-1, Intermediate-2, and High-Risk International Prognostic Scoring System groups. Decitabine is approved in Europe for the treatment of adults with newly diagnosed de novo or secondary AML who are not candidates for standard induction chemotherapy.

Decitabine is believed to exert its antineoplastic effects after phosphorylation and direct incorporation into DNA. Decitabine inhibits DNA methyltransferase, causing hypomethylation of DNA and cellular differentiation or apoptosis. Decitabine-induced hypomethylation in neoplastic cells may restore normal function to genes that are critical for the control of cellular differentiation and proliferation. In rapidly dividing cells, the cytotoxicity of decitabine may also be attributed to the formation of covalent adducts between DNA methyltransferase and compound that has been incorporated into DNA. Non-proliferating cells are relatively insensitive to decitabine.

As used herein the phrase “preparation of a medicament” includes the use of the components of the invention directly as the medicament in addition to their use in any stage of the preparation of such a medicament.

In one preferred embodiment, the decitabine and 1-(2-C-cyano-2-deoxy-β-D-arabino-pentofuranosyl)-N4-palmitoyl cytosine are each administered in a therapeutically effective amount with respect to the individual components; in other words, the decitabine and 1-(2-C-cyano-2-deoxy-β-D-arabino-pentofuranosyl)-N4-palmitoyl cytosine are administered in amounts that would be therapeutically effective even if the components were administered other than in combination.

In another preferred embodiment, the decitabine and 1-(2-C-cyano-2-deoxy-β-D-arabino-pentofuranosyl)-N4-palmitoyl cytosine are each administered in a sub-therapeutic amount with respect to the individual components; in other words, the decitabine and 1-(2-C-cyano-2-deoxy-β-D-arabino-pentofuranosyl)-N4-palmitoyl cytosine are administered in amounts that would be therapeutically ineffective if the components were administered other than in combination.

Preferably, the 1-(2-C-cyano-2-deoxy-β-D-arabino-pentofuranosyl)-N4-palmitoyl cytosine and decitabine interact in a synergistic manner. As used herein, the term “synergistic” means that 1-(2-C-cyano-2-deoxy-β-D-arabino-pentofuranosyl)-N4-palmitoyl cytosine and the decitabine produce a greater effect when used in combination than would be expected from adding the individual effects of the two components. Advantageously, a synergistic interaction may allow for lower doses of each component to be administered to a patient, thereby decreasing the toxicity of chemotherapy, whilst producing and/or maintaining the same therapeutic effect. Thus, in a particularly preferred embodiment, each component can be administered in a sub-therapeutic amount.

Specific Dosing Regimens for AML

Previous studies by the applicant have shown that in AML cell lines, the active metabolite of sapacitabine, CNDAC, is synergistic with hypomethylating agents and the synergy is more apparent if cells are treated with hypomethylating agents first. The present invention relates to the identification of new and previously undefined subgroups of AML patients for which treatment with an alternating regimen of sapacitabine and decitabine is particularly effective.

One aspect of the invention therefore relates to a method of treating a proliferative disorder such as cancer or leukemia in a subject, more specifically AML, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
said method comprising administering to the subject a therapeutically effective amount of (i) sapacitabine, or a metabolite thereof; and (ii) decitabine; in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

The preferred embodiments set forth below apply equally to all aspects of the invention.

The patient subgroup according to the invention is defined in terms of white blood cell (WBC) count and/or antecedent MDS/MPN status, and/or a cytogenetic risk classification for AML according to the US Southwest Oncology Group (SWOG) based on pretreatment karyotypes. An alternating dosing regimen for treating AML with sapacitabine and decitabine is already known in the prior art from Ravandi et al (ibid) and WO 2012/140436. However, these documents are completely silent with regard to the WBC count or SWOG cytogenetic risk classification of the patients. Moreover, there is no teaching or suggestion to indicate that patients having a WBC count below a particular threshold, and/or a cytogenetic risk classification for AML according to the US Southwest Oncology Group (SWOG) that is not unfavourable (e.g. intermediate, favourable, unknown or missing) would be particularly susceptible to treatment with decitabine/sapacitabine combination therapy in accordance with the presently claimed regimen.

The present invention is therefore directed to new subgroups of AML patients treatable with a known combination administered in accordance with a known dosing regimen. The claimed characteristics (<10,000 cells/microliter WBC count and/or not unfavourable SWOG cytogenetic risk classification and/or antecedent MDS, MPN or MDS/MPN) reflect a functional relationship which gives rise to an improved treatment. The combination of a WBC count<10,000 cells/microliter and a not unfavourable SWOG cytogenetic risk classification is particularly preferred. Patients particularly susceptible to treatment with decitabine and sapacitabine can therefore be selected on the basis of these parameters.

In the context of the invention, “and/or” means the subgroup of AML patients fall into one or more, or two or more, or all three of the following categories:
<10,000 cells/microliter WBC count;
not unfavourable SWOG cytogenetic risk classification;
antecedent MDS or antecedent MPN or antecedent MDS/MPN.

In one preferred embodiment, the subgroup of AML patients falls into one of the above categories. In a particularly preferred embodiment, the subgroup of AML patients falls into two of the above categories. In another preferred embodiment, the subgroup of AML patients falls into all three of the above categories.

In one preferred embodiment, the subject has a pretreatment white blood cell (WBC) count of less than about 10,000 cells/microliter. White blood cell count is a standard test used by hospitals which is typically conducted either manually or in automated fashion, according to institutional standard protocols. By way of example, suitable methods are described in Blumenreich [Reference 12] or Shafer [Reference 13], the contents of which are incorporated by reference. The skilled person would be familiar with other suitable methods.

Studies by the applicant have demonstrated that patients with less than about 10,000 cells/microliter WBC before treatment achieve a better median overall survival (mOS), complete response (CR), CR durability and 1-year survival when treated with sapacitabine/decitabine compared to patients with 10,000 cells/microliter compared to treatment with decitabine alone, or compared to treatment with sapacitabine/decitabine in patients with 10,000 WBC. The numerical values for mOS, CR, CR durability and 1-year survival are shown in Table 1.

The data can be analysed using the Kaplan-Meier estimator, also known as the product limit estimator, which is a non-parametric statistic used to estimate the survival function from lifetime data (see Kaplan, E. L.; Meier, P. (1958); “Nonparametric estimation from incomplete observations”; J. Amer. Statist. Assn. 53 (282): 457-481). In medical research, it is often used to measure the fraction of patients living for a certain amount of time after treatment.

As used herein, the numerical value given for the WBC count in each case is considered to have an error margin of ±10%. For example, a WBC count of “10,000” refers to 10,000±1000 cells/microliter. Preferably, the error margin is ±8%, more preferably, ±6%, preferably ±5%, preferably ±4%, preferably ±3%, preferably ±2%, preferably ±1%, more preferably ±0.5% or ±0.2% or ±0.1%. Ultimately, a physician will determine which patients are suitable for treatment according to the invention. Thus, for example, a patient meeting other criteria (e.g. in terms of patient profile, age, health and/or cytogenetic classification), and having a WBC count of less than 11,000 cells/microliter (i.e. below the WBC count threshold when taking into account a 10% error margin) might still be considered suitable for treatment.

Preferably, the subject has a white blood cell (WBC) count of less than about 9000 cells/microliter, more preferably, less than about 8000 cells/microliter, more preferably, less than about 7000 cells/microliter, more preferably, less than about 6000 cells/microliter, even more preferably, less than about 5000 cells/microliter.

In one preferred embodiment, the subject has a white blood cell (WBC) count of less than about 9000 cells/microliter.

In another preferred embodiment, the subject has a white blood cell (WBC) count of less than about 8000 cells/microliter.

In one preferred embodiment, the subject has a white blood cell (WBC) count of less than about 7000 cells/microliter.

In one preferred embodiment, the subject has a white blood cell (WBC) count of less than about 6000 cells/microliter.

In one preferred embodiment, the subject has a white blood cell (WBC) count of less than about 5000 cells/microliter.

In one preferred embodiment, the subject has a pretreatment cytogenetic risk classification for AML according to the US Southwest Oncology Group (SWOG) that is not unfavourable.

Cytogenetic abnormalities are grouped according to published criteria adopted by SWOG [References 1-9, the contents of which are incorporated herein by reference]. Four cytogenetic categories are defined for AML (see Table 6 herein; Slovak et al (Reference 1; Table 1; and page 4076, paragraph bridging columns 1 and 2). Cytogenomic nomenclature is in accordance with standard practice (“An International System for Human Cytogenomic Nomenclature” (2016) ISCN (2016); S. Karger Publishing; ISBN 978-3318058574). As used herein, “abn” refers to an abnormality, “inv” refers to inversion and “del” refers to deletion when compared to the normal chromosome phenotype. The letter p refers to the short arm of the chromosome, the letter q refers to the long arm of the chromosome, and the letter t refers to translocation.

As used herein a cytogenetic risk classification that is “not unfavourable” (or “non-unfavorable”) refers to a cytogenetic risk classification for AML according to the US Southwest Oncology Group (SWOG) that is favorable, intermediate, missing or unknown. Further details of each of these classifications are presented below.

The favorable risk category includes patients with abnormalities (abn) of inv(16)/t(16;16)/del(16q) or t(15;17) with/without any additional abnormalities, or t(8;21) without either a del(9q) or being part of a complex karyotype. The presence of a del(9q) in patients with t(8;21) leukemia has been reported as a poor risk indicator requiring more aggressive treatment [Reference 8].

The intermediate risk category includes patients characterized by one or more of +8, −Y, +6, del(12p), or normal karyotype.

The unfavorable risk category is defined by the presence of one or more of del(5q)/−5, −7(del(7q),abn 3q, 9q, 11q, 20q, 21q, 17p, t(6;9), t(9:22) and complex karyotypes (≥3 unrelated abnormalities). More preferably, the unfavorable risk category is defined by the presence of one or more of −5/del(5q), −7/del(7q), inv(3q), abn 11q, 20q, or 21q, del(9q), t(6;9), t(9;22), abn 17p, and complex karyotypes (≥3 unrelated abnormalities).

The unknown risk category includes cytogenetic aberrations considered to have unknown prognostic significance because of their low frequency in AML.

In some cases of AML, multiple unrelated cytogenetic abnormalities will be seen in a single karyotype. If the number of abnormalities is sufficient, such cases are defined as having “complex” cytogenetics or a “complex karyotype” [Reference 20; the contents of which are incorporated by reference]. In the case of SWOG, three or more abnormalities are required for this definition.

As used herein, the term “normal karyotype” refers to AML without any cytogenetic abnormalities.

Studies by the applicant have demonstrated that patients for whom the cytogenetic risk classification for AML by the SWOG is not unfavorable (for example, intermediate/favorable/unknown/missing) achieve a better mOS and better CR and 1-year survival rates when treated with sapacitabine/decitabine compared to decitabine, or compared to treatment with sapacitabine/decitabine in patients with unfavourable cytogenetics by SWOG. See Table 2.

In one preferred embodiment, the subject has a cytogenetic risk classification for AML according to the US Southwest Oncology Group (SWOG) (see Reference 1) that is intermediate. For example, preferably, the patient is characterized by one or more of the following: +8, −Y, +6, del(12p), or normal karyotype. In one preferred embodiment, the patient is characterized by +8. In one preferred embodiment, the patient is characterized by +6. In another preferred embodiment, the patient is characterized by −Y. In another preferred embodiment, the patient is characterized by del(12p). In another preferred embodiment, the patient is characterized by having a normal karyotype.

In another preferred embodiment, the subject has a cytogenetic risk classification for AML according to the US Southwest Oncology Group (SWOG) (see Reference 1) that is favourable. For example, preferably, the patient is characterized by one or more of the following: inv(16)/t(16;16)/del(16q) or t(15;17) with or without additional secondary abnormalities, or t(8;21) lacking del(9q) or being part of a complex karyotype. Additional abnormalities may include, for example, −5/5q or −7/7q. In one preferred embodiment, the patient is characterized by inv(16)/t(16;16)/del(16q) with/without additional abnormalities. In another preferred embodiment, the patient is characterized by t(15;17) with/without additional abnormalities. In another preferred embodiment, the patient is characterized by t(8;21) lacking del(9q) or being part of a complex karyotype. In one preferred embodiment, the patient is characterized by inv(16)/t(16;16) or t(15;17) and complex abnormalities.

In another preferred embodiment, the subject has a cytogenetic risk classification for AML according to the US Southwest Oncology Group (SWOG) (see Reference 1) that is unknown. For example, preferably, the patient is characterized by cytogenetic aberrations considered to have unknown prognostic significance.

In another preferred embodiment, the subject has a cytogenetic risk classification for AML according to the US Southwest Oncology Group (SWOG) (see Reference 1) that is missing. Preferably, the subject has a cytogenetic risk classification according to the US Southwest Oncology Group (SWOG) (see Reference 1) that is intermediate, favourable or unknown, more preferably intermediate or favourable.

In one particularly preferred embodiment, the subject has a white blood cell (WBC) count of less than about 10,000 cells/microliter and a cytogenetic risk classification for AML according to the US Southwest Oncology Group (SWOG) that is not unfavourable.

Studies by the applicant have demonstrated that patients for whom the cytogenetic risk classification by SWOG is not unfavorable (for example, intermediate/favorable/unknown/missing) and who have <10,000 WBC before treatment achieve a better mOS and better CR and 1-year survival when treated with sapacitabine/decitabine compared to decitabine only, or compared to treatment with sapacitabine/decitabine in patients with 10,000 WBC and unfavourable cytogenetics by SWOG. This combination of characteristics is particularly favourable. See Table 3.

In one highly preferred embodiment, the subject has a cytogenetic risk classification for AML according to the US Southwest Oncology Group (SWOG) that is intermediate and a WBC count of less than about 10,000 cells/microliter.

In another highly preferred embodiment, the subject has a cytogenetic risk classification according to the US Southwest Oncology Group (SWOG) that is favourable and a WBC count of less than about 10,000 cells/microliter.

In another highly preferred embodiment, the subject has a cytogenetic risk classification according to the US Southwest Oncology Group (SWOG) that is unknown and a WBC count of less than about 10,000 cells/microliter.

In another highly preferred embodiment, the subject has a cytogenetic risk classification according to the US Southwest Oncology Group (SWOG) that is missing and a WBC count of less than about 10,000 cells/microliter.

In another preferred embodiment, the subject falls within a classification selected from antecedent myelodysplastic syndrome (MDS), antecedent myeloproliferative neoplasm (MPN), and antecedent myelodysplastic/myeloproliferative neoplasm (MDS/MPN).

As used herein, the term “antecedent MDS” means previously diagnosed MDS at any time prior to the diagnosis of AML.

As used herein, the term “antecedent MPN” means previously diagnosed MPN at any time prior to the diagnosis of AML.

As used herein, the term “antecedent MDS/MPN” means previously diagnosed MPN/MDS at any time prior to the diagnosis of AML.

The WHO categories of Myeloproliferative neoplasms (MPN), Myelodysplastic/myeloproliferative neoplasms (MDS/MPN) and Myelodysplastic syndrome (MDS) which comprise the antecedent MDS or MPN diseases are shown in Table 7 herein, and are as defined in Vardiman et al, (Blood, 30 July 2009, Volume 114, Number 5, pages 937-951; “The 2008 revision of the WHO classification of myeloid neoplasms and acute leukemia: rationale and important changes”; Reference 23; see in particular, Table 2), and Swerdlow S. H., Campo E., Harris N. L., et al., editors “WHO Classification of Tumours of Haematopoietic and Lymphoid Tissues”, Lyon, France: IARC; 2008; Reference 24), the contents of both of which are herein incorporated by reference.

In one preferred embodiment, the subject falls within the classification of antecedent MDS. MDS includes refractory cytopenia with unilineage dysplasia, refractory anemia, refractory neutropenia, refractory thrombocytopenia, refractory anemia with ring sideroblasts, refractory cytopenia with multilineage dysplasia, refractory anemia with excess blasts, myelodysplastic syndrome with isolated del(5q), myelodysplastic syndrome (unclassifiable), childhood myelodysplastic syndrome, and refractory cytopenia of childhood (provisional entry); (see Table 7).

The “minimal” morphologic criteria for the diagnosis of MDS remain similar to those stated in the 3rd edition of the WHO Classification of Tumours of the Hematopoietic and Lymphoid Tissue (2001): in the appropriate clinical setting, at least 10% of the cells of at least one myeloid bone marrow lineage (erythroid, granulocytic, megakaryocytic) must show unequivocal dysplasia for the lineage to be considered as dysplastic [References 25, 26, 27]. Causes of secondary dysplasia as well as congenital abnormalities such as congenital dyserythropoietic anemia should be excluded before a diagnosis of MDS is rendered. If, however, a patient with clinical and other laboratory features consistent with MDS has inconclusive morphologic features, a presumptive diagnosis of MDS can be made if a specific clonal chromosomal abnormality, listed in Table 8 herein, is present.

In one preferred embodiment, the subject falls within the classification of antecedent MPN. MPN includes chronic myelogenous leukemia (BCR-ABL1-positive), chronic neutrophilic leukemia, polycythemia vera, primary myelofibrosis, essential thrombocythemia, chronic eosinophilic leukemia (not otherwise specified), mastocytosis, myeloproliferative neoplasms (unclassifiable); (see Table 7). Criteria for polycythemia vera, essential thrombocythemia and primary myelofibrosis are as defined in Tables 9, 10 and 11 respectively (in accordance with Tables 3, 4, 5 of Vardiman et al [Reference 23], the contents of which are incorporated by reference.

In some cases, the genetic abnormality, such as the BCR-ABL1 fusion gene in CML, is associated with consistent clinical, laboratory, and/or morphologic features that allow the genetic abnormality to be used as a major criterion for diagnosis of MPN. Other abnormalities, such as mutated JAK2 or KIT, are not specific for any single MPN but provide proof that the proliferation is clonal. The most commonly recognized mutation in BCR-ABL1-negative MPN is JAK2 V617F [References 28, 37-39]. This mutation is found in more than 90% of patients with polycythemia vera (PV) and in nearly one-half of those with primary myelofibrosis (PMF) or essential thrombocythemia (ET) [References 37, 28]. In the few PV patients who lack this mutation, a similar activating JAK2 exon 12 mutation may be found, [Reference 40] and a small proportion of patients with PMF and ET who lack mutated JAK2 may instead demonstrate activating mutations of MPL, such as MPL W515K or MPL W515L [Reference 41].

In one preferred embodiment, the subject falls within the classification of antecedent MDS/MPN. The MDS/MPN category includes myeloid neoplasms with clinical, laboratory, and morphologic features that overlap MDS and MPN. This subgroup includes chronic myelomonocytic leukemia (CMML), atypical chronic myeloid leukemia (aCML) (BCR-ABL1-negative), juvenile myelomonocytic leukemia (JMML), and a provisional entity within the MDS/MPN unclassifiable group, refractory anemia with ring sideroblasts and thrombocytosis (RARS-T); (see Table 7). A few cases of CMML and aCML have been reported to demonstrate JAK2 mutations [References 28, 29, 30] but the proliferative aspects of most cases are related to aberrancies in the RAS/MAPK signaling pathways. In JMML, nearly 75% of patients demonstrate mutually exclusive mutations of PTPN11, NRAS or KRAS, or NF1, all of which encode signaling proteins in RAS dependent pathways [References 31, 32]. Approximately 30% to 40% of cases of CMML and aCML also exhibit NRAS or KRAS mutations [References 33-36].

For patients with antecedent MDS/MPN, median overall survival was higher on the sapacitabine/decitabine treatment vs. decitabine treatment (6.4 months vs. 5.0 months), as was CR rate (16.7% vs. 5.7%). Median overall survival was also higher for patients with antecedent MDS/MPN treated with sapacitabine/decitabine vs the patients without antecedent MDS/MPN treated with sapacitabine/decitabine (6.4 months vs 5.9 months), as was CR duration (9.5 months vs 8.5 months). In addition, for the patients treated with sapacitabine/decitabine with antecedent MDS/MPN vs those without antecedent MDS/MPN 1-year survival was greater (34.8% vs 33.1%). See Table 4.

In one preferred embodiment, the subject falls within a classification selected from antecedent myelodysplastic syndrome (MDS), antecedent myeloproliferative neoplasm (MPN), and antecedent myelodysplastic/myeloproliferative neoplasm (MDS/MPN), and has a white blood cell (WBC) count of less than about 10,000 cells/microliter. This combination of characteristics is particularly favourable. See Table 5.

For patients with antecedent MDS/MPN and <10,000 WBC, median overall survival was higher on the sapacitabine/decitabine treatment vs. decitabine treatment (6.8 months vs. 4.9 months), as was CR rate (19.4% vs. 2.5%). Median overall survival was also higher for the patients with antecedent MDS/MPN and <10,000 WBC vs those without antecedent MDS/MPN and ≥10,000 WBC, both treated with sapacitabine/decitabine (6.8 months vs 3.8 months), as was CR rate (19.4% vs 5.6%). In addition, for the patients treated with sapacitabine/decitabine with antecedent MDS/MPN and <10,000 WBC vs those without antecedent MDS/MPN and ≥10,000 WBC 1-year survival was greater (41.7% vs 11.1%).

In one preferred embodiment, the subject falls within a classification of antecedent myelodysplastic syndrome (MDS), and has a white blood cell (WBC) count of less than about 10,000 cells/microliter.

In another preferred embodiment, the subject falls within a classification of antecedent myeloproliferative neoplasm (MPN), and has a white blood cell (WBC) count of less than about 10,000 cells/microliter.

In another preferred embodiment, the subject falls within a classification of antecedent myelodysplastic/myeloproliferative neoplasm (MDS/MPN), and has a white blood cell (WBC) count of less than about 10,000 cells/microliter.

In another preferred embodiment, the subject falls within a classification selected from antecedent myelodysplastic syndrome (MDS), antecedent myeloproliferative neoplasm (MPN) and antecedent myelodysplastic/myeloproliferative neoplasm (MDS/MPN), and has a cytogenetic risk classification for AML according to the US Southwest Oncology Group (SWOG) that is not unfavourable, i.e. favorable, intermediate, unknown or missing. In one preferred embodiment, the cytogenetic risk classification according to the US Southwest Oncology Group (SWOG) is favourable. In one preferred embodiment, the cytogenetic risk classification according to the US Southwest Oncology Group (SWOG) is intermediate. In one preferred embodiment, the cytogenetic risk classification according to the US Southwest Oncology Group (SWOG) is unknown. In one preferred embodiment, the cytogenetic risk classification according to the US Southwest Oncology Group (SWOG) is missing.

In another preferred embodiment, the subject falls within a classification of antecedent myelodysplastic syndrome (MDS), and has a cytogenetic risk classification for AML according to the US Southwest Oncology Group (SWOG) that is not unfavourable. In one preferred embodiment, the cytogenetic risk classification according to the US Southwest Oncology Group (SWOG) is favourable. In one preferred embodiment, the cytogenetic risk classification according to the US Southwest Oncology Group (SWOG) is intermediate. In one preferred embodiment, the cytogenetic risk classification according to the US Southwest Oncology Group (SWOG) is unknown. In one preferred embodiment, the cytogenetic risk classification according to the US Southwest Oncology Group (SWOG) is missing.

In another preferred embodiment, the subject falls within a classification of antecedent myeloproliferative neoplasm (MPN), and has a cytogenetic risk classification for AML according to the US Southwest Oncology Group (SWOG) that is not unfavourable. In one preferred embodiment, the cytogenetic risk classification according to the US Southwest Oncology Group (SWOG) is favourable. In one preferred embodiment, the cytogenetic risk classification according to the US Southwest Oncology Group (SWOG) is intermediate. In one preferred embodiment, the cytogenetic risk classification according to the US Southwest Oncology Group (SWOG) is unknown. In one preferred embodiment, the cytogenetic risk classification according to the US Southwest Oncology Group (SWOG) is missing.

In another preferred embodiment, the subject falls within a classification of antecedent myelodysplastic/myeloproliferative neoplasm (MDS/MPN), and has a cytogenetic risk classification for AML according to the US Southwest Oncology Group (SWOG) that is not unfavourable. In one preferred embodiment, the cytogenetic risk classification according to the US Southwest Oncology Group (SWOG) is favourable. In one preferred embodiment, the cytogenetic risk classification according to the US Southwest Oncology Group (SWOG) is intermediate. In one preferred embodiment, the cytogenetic risk classification according to the US Southwest Oncology Group (SWOG) is unknown. In one preferred embodiment, the cytogenetic risk classification according to the US Southwest Oncology Group (SWOG) is missing.

In another preferred embodiment, the subject falls within a classification selected from antecedent myelodysplastic syndrome (MDS), antecedent myeloproliferative neoplasm (MPN), and antecedent myelodysplastic/myeloproliferative neoplasm (MDS/MPN), and has a white blood cell (WBC) count of less than about 10,000 cells/microliter, and has a cytogenetic risk classification for AML according to the US Southwest Oncology Group (SWOG) that is not unfavourable, i.e. favorable, intermediate, unknown or missing.

In another preferred embodiment, the subject falls within a classification of antecedent myelodysplastic syndrome (MDS), and has a white blood cell (WBC) count of less than about 10,000 cells/microliter, and has a cytogenetic risk classification for AML according to the US Southwest Oncology Group (SWOG) that is not unfavourable, i.e. favorable, intermediate, unknown or missing.

In another preferred embodiment, the subject falls within a classification of antecedent myeloproliferative neoplasm (MPN), and has a white blood cell (WBC) count of less than about 10,000 cells/microliter, and has a cytogenetic risk classification for AML according to the US Southwest Oncology Group (SWOG) that is not unfavourable, i.e. favorable, intermediate, unknown or missing.

In another preferred embodiment, the subject falls within a classification of antecedent myelodysplastic/myeloproliferative neoplasm (MDS/MPN), and has a white blood cell (WBC) count of less than about 10,000 cells/microliter, and has a cytogenetic risk classification for AML according to the US Southwest Oncology Group (SWOG) that is not unfavourable, i.e. favorable, intermediate, unknown or missing.

In one preferred embodiment, the second treatment cycle comprises administering a therapeutically effective amount of sapacitabine.

The sequential administration of decitabine and sapacitabine in alternating cycles in accordance with the presently claimed dosing regimen maximizes the efficacy of both drugs and minimizes overlapping myelosuppression.

The first and second treatment cycles are repeated sequentially with rest periods between sequential cycles, i.e. there is a rest period between the last day of decitabine administration and the first day of the second treatment cycle; likewise there is a rest period between the last day of sapacitabine administration and the first day of the next (first) treatment cycle. Preferably, the rest period is sufficient so as to resolve any treatment-related toxicities.

As used herein, treatment-related toxicities include myelosuppression and its associated complications. Myelosuppression is a term commonly used in the art and refers specifically to a reduction in the ability of the bone marrow to produce red blood cells, platelets and white blood cells. Myelosuppression causes anemia (low levels of red blood cells), neutropenia (low levels of neutrophils, a type of white blood cell) and thrombocytopenia (low levels of platelets). Associated complications of myelosuppression include fatigue (due to anemia), infections (due to neutropenia) and bruising/bleeding (due to thrombocytopenia).

In one preferred embodiment, the first treatment cycle comprises administering a therapeutically effective amount of decitabine for 5 consecutive days followed by a rest period of from 3 to 5 weeks, or until treatment-related toxicities are resolved, whichever is longer.

In one preferred embodiment, the first treatment cycle comprises administering a therapeutically effective amount of decitabine for 5 days followed by a rest period of 3 to 5 weeks.

In a more preferred embodiment, the first treatment cycle comprises administering a therapeutically effective amount of decitabine for 5 days followed by a rest period of 3 weeks.

In one preferred embodiment, the first treatment cycle comprises administering a therapeutically effective amount of decitabine for 10 consecutive days followed by a rest period of from 3 to 5 weeks, or until treatment-related toxicities are resolved, whichever is longer.

In another preferred embodiment, the first treatment cycle comprises administering a therapeutically effective amount of decitabine for 10 days followed by a rest period of 4 weeks.

In one preferred embodiment, the second treatment cycle comprises administering a therapeutically effective amount of sapacitabine or metabolite thereof for 3 consecutive days per week, for 2 weeks, followed by a rest period of 2 to 4 weeks.

In a more preferred embodiment, the second treatment cycle comprises administering a therapeutically effective amount of sapacitabine or metabolite thereof for 3 consecutive days per week, for 2 weeks, followed by a rest period of 2 weeks.

In one preferred embodiment, the method comprises two or more of each treatment cycle, more preferably, three or more, four or more, or five or more of each treatment cycle.

In one highly preferred embodiment, the method comprises four or more of each treatment cycle.

In one highly preferred embodiment, the method comprises two to four of each treatment cycle.

In one highly preferred embodiment, the method comprises administering decitabine for 5 consecutive days of a 4-week cycle (odd cycles) alternating with administering sapacitabine for three days per week for two weeks of a 4-week cycle (even cycles).

In one preferred embodiment, the decitabine is administered intravenously.

In one preferred embodiment, the decitabine is administered in a dose of from about 10 to 20 mg/m² per day.

In a more preferred embodiment, the decitabine is administered in a dose of about 20 mg/m² per day. In certain preferred embodiments, the decitabine dosage may be tailored to individual patients within the same schedule in order to mitigate side effects. For example, in certain preferred embodiments the decitabine dosage may be reduced (typically in 5 mg/m² increments) from a starting dose of about 20 mg/m² per day, to about 15mg/m² per day, or to about 10 mg/m² per day.

In one preferred embodiment, the decitabine is administered over a period of up to 3 hours per day, more preferably over a period of up to 2 hours per day, even more preferably over a period of about 1 hour per day. Preferably, the decitabine is administered by intravenous infusion over a period of about 1 hour.

In one preferred embodiment, the first treatment cycle comprises administering a therapeutically effective amount of decitabine in a dosage of about 20 mg/m² for 10 days, followed by a rest period of 4 weeks. In one preferred embodiment, the first treatment cycle comprises administering a therapeutically effective amount of decitabine in a dosage of about 20 mg/m² for 5 days, followed by a rest period of 4 weeks.

In one preferred embodiment, the sapacitabine or metabolite thereof is administered orally.

In one preferred embodiment, the sapacitabine or metabolite thereof is administered in a dose of about 100-400 mg b.i.d., more preferably from about 250-300 mg b.i.d.

In a more preferred embodiment, the sapacitabine or metabolite thereof is administered in a dose of about 300 mg b.i.d. In certain preferred embodiments, the sapacitabine dosage may be tailored to individual patients within the same schedule in order to mitigate side effects. For example, in certain preferred embodiments the sapacitabine dosage may be reduced (typically in 50 mg increments) from a starting dose of about 300 mg b.i.d. to about 250 mg b.i.d., or to about 200 mg b.i.d., or to about 150 mg b.i.d., or to about 100 mg b.i.d.

In one preferred embodiment, the subject is an adult, more preferably, an elderly subject. As used herein, the term “elderly subject” refers to a subject of 60 years of age or over. More preferably, the subject is 65 years of age or over, even more preferably, 70 years of age or over, more preferably still, 75 years of age or over.

In one preferred embodiment, the subject is not considered a suitable candidate for intensive induction therapy. Intensive induction therapy involves an initial treatment phase in which high dosages of therapeutic agent are administered to a subject that has received no prior treatment for AML, with the aim of achieving remission. Intensive induction therapy is believed to target naïve tumour cells possibly different from their counterparts in remission in terms of their kinetic status and sensitivity. However, not all subjects are suitable for intensive induction therapy, for example, elderly patients, or those in poor general health or having a poor level of general fitness. Patients for whom the treatment of choice is low intensity therapy are typically selected by investigator assessment. There is no approved scoring system and it is primarily a patient by patient judgement made by the physician. The investigator will take into account a number of factors, including, but not limited to, the patient's age, their overall quality of health, the presence of any non-cancer significant illnesses and/or the characteristics of their disease, such as the presence of certain mutations [see References 15-19; the contents of which are incorporated by reference].

In one preferred embodiment, the subject is a newly diagnosed AML subject. As used herein, newly diagnosed AML refers to a subject who is treatment naïve, i.e. a histologically or pathologically confirmed diagnosis of AML based on WHO classification which has not been treated by any systemic therapy administered orally, intravenously or subcutaneously (except hydroxyurea) [see References 10, 11; the contents of which are incorporated by reference].

In one preferred embodiment, the patient has not been previously treated with a hypomethylating agent for prior myelodysplastic syndrome (MDS) or myeloproliferative disease (MPD; also known as myeloproliferative neoplasm or MPN).

More preferably, the subject is not considered a candidate for intensive induction therapy and is a treatment naïve (newly diagnosed) AML subject.

A further aspect of the invention relates to a method of treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
said method comprising administering to a subject a therapeutically effective amount of (i) sapacitabine; and (ii) decitabine; in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

In one highly preferred embodiment, the dosing regimen comprises administering decitabine at 20 mg/m² per day for 5 consecutive days of a 4-week cycle (odd cycles) and sequentially sapacitabine at 300 mg orally twice per day for three days per week for two weeks of a 4-week cycle (even cycles).

A further aspect of the invention relates to a method of treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
said method comprising administering to a subject a therapeutically effective amount of (i) sapacitabine; and (ii) decitabine; in accordance with a dosing regimen comprising at least one first treatment cycle and at least one second treatment cycle,
wherein said first treatment cycle comprises administering decitabine intravenously in a dose of about 20 mg/m² per day for 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

In one highly preferred embodiment, the dosing regimen comprises administering decitabine at 20 mg/m² per day for 10 consecutive days of a 4-week cycle (odd cycles) and sequentially sapacitabine at 300 mg orally twice per day for three days per week for two weeks of a 4-week cycle (even cycles).

A further aspect of the invention relates to (i) sapacitabine, or a metabolite thereof; and (ii) decitabine; for use in treating AML in a subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein the sapacitabine, or a metabolite thereof, and the decitabine are administered in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

Another aspect of the invention relates to (i) sapacitabine, or a metabolite thereof; and (ii) decitabine; for use in treating AML in an elderly subject, wherein said subject:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN); and
wherein the sapacitabine, or metabolite thereof, and decitabine, are administered in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer.

A further aspect of the invention relates to the use of (i) sapacitabine, or a metabolite thereof; and (ii) decitabine; in the preparation of a medicament for treating AML in a subject, wherein said subject has:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN); and
wherein the sapacitabine, or a metabolite thereof, and the decitabine are administered in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

Another aspect of the invention relates to the use of (i) sapacitabine, or a metabolite thereof; and (ii) decitabine; in the preparation of a medicament for treating AML in an elderly subject, wherein said subject has:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);

and wherein the sapacitabine, or metabolite thereof, and decitabine, are administered in accordance with a dosing regimen comprising at least one first treatment cycle and at least one second treatment cycle,
wherein said first treatment cycle comprises administering decitabine intravenously in a dose of about 20 mg/m² per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

Further aspects of the invention relate to a method of selecting subject suitable for treatment with sapacitabine and decitabine in accordance with the dosing regimen described herein, said method comprising measuring the WBC count in a sample obtained from the subject, and/or determining the cytogenetic risk classification according to the US Southwest Oncology Group (SWOG) of a sample obtained from the subject.

Kit of Parts

A further aspect of the invention relates to a kit of parts comprising:

(i) sapacitabine, or a metabolite thereof;

(ii) decitabine; and

(iii) instructions for administering sapacitabine, or a metabolite thereof, and decitabine to a subject, in accordance with a dosing regimen comprising at least one first treatment cycle and at least one second treatment cycle, wherein said subject has:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

Another aspect of the invention relates to a kit of parts comprising:

(i) sapacitabine, or a metabolite thereof;

(ii) decitabine; and

(iii) instructions for administering sapacitabine, or a metabolite thereof, and decitabine to a subject in accordance with a dosing regimen comprising at least one first treatment cycle and at least one second treatment cycle, wherein said subject has:

(I) has a white blood cell (WBC) count of less than about 10,000 cells/microliter, and/or

(II) has a cytogenetic risk classification according to the US Southwest Oncology Group (SWOG) that is not unfavourable, and/or

(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein said first treatment cycle comprises administering decitabine intravenously in a dose of about 20 mg/m² for 5 to 10 consecutive days followed by a 3 to 5 week rest period, or until treatment-related toxicities are resolved, whichever is longer; and
wherein said second treatment cycle comprises administering sapacitabine orally in a dose of about 300 mg for 3 consecutive days per week, for 2 weeks followed by a 2 to 4 week rest period, or until treatment-related toxicities are resolved, whichever is longer. Preferably, the kit of parts is for use in treating ALM in a subject, preferably an elderly subject.

Metabolite

As used herein, the term “metabolite” encompasses chemically modified entities that are produced by metabolism of 1-(2-C-cyano-2-deoxy-β-D-arabino-pentofuranosyl)-N4-palmitoyl cytosine (sapacitabine).

In one particularly preferred embodiment of the invention, the metabolite of 1-(2-C-cyano-2-deoxy-β-D-arabino-pentofuranosyl)-N4-palmitoyl cytosine is 2′-C-cyano-2′-deoxy-1-β-D-arabino-pentofuranosyl cytosine (CNDAC).

In another particularly preferred embodiment of the invention, 1-(2-C-cyano-2-deoxy-β-D-arabino-pentofuranosyl)-N4-palmitoyl cytosine is metabolized intracellularly to the active metabolite CNDAC-triphosphate (CNDACTP), a process involving both the cleavage of the palmitoyl moiety and activation to CNDACTP by the action of nucleoside kinases.

Salts/Esters

The agents of the present invention can be present as salts or esters, in particular pharmaceutically acceptable salts or esters.

Pharmaceutically acceptable salts of the agents of the invention include suitable acid addition or base salts thereof. A review of suitable pharmaceutical salts may be found in Berge et al, J Pharm Sci, 66, 1-19 (1977). Salts are formed, for example with strong inorganic acids such as mineral acids, e.g. sulphuric acid, phosphoric acid or hydrohalic acids; with strong organic carboxylic acids, such as alkanecarboxylic acids of 1 to 4 carbon atoms which are unsubstituted or substituted (e.g., by halogen), such as acetic acid; with saturated or unsaturated dicarboxylic acids, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid.

Esters are formed either using organic acids or alcohols/hydroxides, depending on the functional group being esterified. Organic acids include carboxylic acids, such as alkanecarboxylic acids of 1 to 12 carbon atoms which are unsubstituted or substituted (e.g., by halogen), such as acetic acid; with saturated or unsaturated dicarboxylic acid, for example oxalic, malonic, succinic, maleic, fumaric, phthalic or tetraphthalic; with hydroxycarboxylic acids, for example ascorbic, glycolic, lactic, malic, tartaric or citric acid; with aminoacids, for example aspartic or glutamic acid; with benzoic acid; or with organic sulfonic acids, such as (C₁-C₄)-alkyl- or aryl-sulfonic acids which are unsubstituted or substituted (for example, by a halogen) such as methane- or p-toluene sulfonic acid. Suitable hydroxides include inorganic hydroxides, such as sodium hydroxide, potassium hydroxide, calcium hydroxide, aluminium hydroxide. Alcohols include alkanealcohols of 1-12 carbon atoms which may be unsubstituted or substituted, (e.g. by a halogen).

Enantiomers/Tautomers

The invention also includes where appropriate all enantiomers and tautomers of the agents. Those skilled in the art will recognise compounds that possess optical properties (one or more chiral carbon atoms) or tautomeric characteristics. The corresponding enantiomers and/or tautomers may be isolated/prepared by methods known in the art.

Stereo and Geometric Isomers

Some of the agents of the invention may exist as stereoisomers and/or geometric isomers—e.g. they may possess one or more asymmetric and/or geometric centres and so may exist in two or more stereoisomeric and/or geometric forms. The present invention contemplates the use of all the individual stereoisomers and geometric isomers of those inhibitor agents, and mixtures thereof. The terms used in the claims encompass these forms, provided said forms retain the appropriate functional activity (though not necessarily to the same degree).

Isotopic Variations

The present invention also includes all suitable isotopic variations of the agent or pharmaceutically acceptable salts thereof. An isotopic variation of an agent of the present invention or a pharmaceutically acceptable salt thereof is defined as one in which at least one atom is replaced by an atom having the same atomic number but an atomic mass different from the atomic mass usually found in nature. Examples of isotopes that can be incorporated into the agent and pharmaceutically acceptable salts thereof include isotopes of hydrogen, carbon, nitrogen, oxygen, phosphorus, sulphur, fluorine and chlorine such as ²H, ³H, ¹³C, ¹⁴C, ¹⁵N, ¹⁷O, ¹⁸O, ³¹P, ³²P, ³⁵S, ¹⁸F and ³⁶Cl, respectively. Certain isotopic variations of the agent and pharmaceutically acceptable salts thereof, for example, those in which a radioactive isotope such as ³H or ¹⁴C is incorporated, are useful in drug and/or substrate tissue distribution studies. Tritiated, i.e., ³H, and carbon-14, i.e., ¹⁴C, isotopes are particularly preferred for their ease of preparation and detectability. Further, substitution with isotopes such as deuterium, i.e., ²H, may afford certain therapeutic advantages resulting from greater metabolic stability, for example, increased in vivo half-life or reduced dosage requirements and hence may be preferred in some circumstances. Isotopic variations of the agent of the present invention and pharmaceutically acceptable salts thereof of this invention can generally be prepared by conventional procedures using appropriate isotopic variations of suitable reagents.

Solvates

The present invention also includes solvate forms of the agents of the present invention. The terms used in the claims encompass these forms.

Polymorphs

The invention furthermore relates to agents of the present invention in their various crystalline forms, polymorphic forms and (an)hydrous forms. It is well established within the pharmaceutical industry that chemical compounds may be isolated in any of such forms by slightly varying the method of purification and or isolation form the solvents used in the synthetic preparation of such compounds.

Prodrugs

The invention further includes agents of the present invention in prodrug form. Such prodrugs are generally compounds wherein one or more appropriate groups have been modified such that the modification may be reversed upon administration to a human or mammalian subject. Such reversion is usually performed by an enzyme naturally present in such subject, though it is possible for a second agent to be administered together with such a prodrug in order to perform the reversion in vivo. Examples of such modifications include esters (for example, any of those described above), wherein the reversion may be carried out be an esterase etc. Other such systems will be well known to those skilled in the art.

Administration

The pharmaceutical compositions of the present invention may be adapted for oral, rectal, vaginal, parenteral, intramuscular, intraperitoneal, intraarterial, intrathecal, intrabronchial, subcutaneous, intradermal, intravenous, nasal, buccal or sublingual routes of administration.

For oral administration, particular use is made of compressed tablets, pills, tablets, gellules, drops, and capsules. Preferably, these compositions contain from 1 to 2000 mg and more preferably from 50-1000 mg, of active ingredient per dose.

Other forms of administration comprise solutions or emulsions which may be injected intravenously, intraarterially, intrathecally, subcutaneously, intradermally, intraperitoneally or intramuscularly, and which are prepared from sterile or sterilisable solutions. The pharmaceutical compositions of the present invention may also be in form of suppositories, pessaries, suspensions, emulsions, lotions, ointments, creams, gels, sprays, solutions or dusting powders.

An alternative means of transdermal administration is by use of a skin patch. For example, the active ingredient can be incorporated into a cream consisting of an aqueous emulsion of polyethylene glycols or liquid paraffin. The active ingredient can also be incorporated, at a concentration of between 1 and 10% by weight, into an ointment consisting of a white wax or white soft paraffin base together with such stabilisers and preservatives as may be required.

Injectable forms may contain between 10-1000 mg, preferably between 10-500 mg, of active ingredient per dose.

Compositions may be formulated in unit dosage form, i.e., in the form of discrete portions containing a unit dose, or a multiple or sub-unit of a unit dose.

In a particularly preferred embodiment, the combination or pharmaceutical composition of the invention is administered intravenously.

Dosage

A person of ordinary skill in the art can easily determine an appropriate dose of one of the instant compositions to administer to a subject without undue experimentation. Typically, a physician will determine the actual dosage which will be most suitable for an individual patient and it will depend on a variety of factors including the activity of the specific compound employed, the metabolic stability and length of action of that compound, the age, body weight, general health, sex, diet, mode and time of administration, rate of excretion, drug combination, the severity of the particular condition, and the individual undergoing therapy. The dosages disclosed herein are exemplary of the average case. There can of course be individual instances where higher or lower dosage ranges are merited, and such are within the scope of this invention.

Depending upon the need, the agent may be administered at a dose of from 0.1 to 30 mg/kg body weight, such as from 2 to 20 mg/kg, more preferably from 0.1 to 1 mg/kg body weight.

By way of guidance, 1-(2-C-cyano-2-deoxy-β-D-arabino-pentafuranosyl)-N4-palmitoyl cytosine (sapactibine) is typically administered in accordance with a physician's direction at total dosages of between 100 mg and 800 mg per day. Preferably, the dose is administered orally. The doses can be given 5 days a week for 4 weeks, or 3 days a week for 4 weeks. Dosages and frequency of application are typically adapted to the general medical condition of the patient and to the severity of the adverse effects caused, in particular to those caused to the hematopoietic, hepatic and to the renal system. The total daily dose can be administered as a single dose or divided into separate dosages administered two, three or four time a day.

The DNA methyltransferase inhibitor decitabine (Dacogen®) is typically administered subcutaneously or intravenously in accordance with a physician's direction. By way of guidance, the recommended decitabine dose for its approved use is 15 mg/m² administered by continuous intravenous infusion over 3 h repeated every 8 h for 3 days (decitabine clinical label; Fenaux P. (2005) Nature Clinical Practice, 2, S36-44). This cycle is preferably repeated every 6 weeks. Patients with advanced solid tumours typically receive a 72 h infusion of decitabine at 20-30 mg/m²/day.

For the purposes of the present invention, decitabine is preferably administered at a dose of 20 mg/m² by continuous intravenous infusion over 1 hour repeated daily for 5 days. The cycle is repeated every 4 weeks (see FDA approved drug label for decitabine).

The present invention is further described by way of the following non-limiting examples.

### EXAMPLES

Materials & Methods

Reagents

CNDAC was prepared in accordance with the methodology set forth in EP 535231B (Sankyo Company Limited). Sapacitabine was prepared in accordance with the methodology described in EP 536936B (Sankyo Company Limited). Sapacitabine is formulated as a liquid fill capsule in Miglyol812N Ph. Eur/GRAS, in accordance with Example 3 of WO2007072061 (Cyclacel Limited).

Decitabine (approved as Dacogen®) is commercially available from a number of sources including Otsuka Pharm Co Limited (NDA #021790), Sandoz Inc. (ANDA #202969), Dr Reddys Labs Limited (ANDA #203131), Accord Healthcare (ANDA #203475), Pharmascience Inc. (#204607), Sun Pharma Global (NDA #205582) and Chemi SPA (ANDA #206033).

Decitabine is formulated in accordance with the label for Dacogen®, i.e. as a sterile, lyophilised white to almost white powder for injection, in a single dose vial. Dacogen is administered by intravenous infusion. Each vial of powder for concentrate for solution for infusion contains 50 mg decitabine. Each vial contains 0.5 mmol potassium (Potassium dihydrogen phosphate; E340) and 0.29 mmol sodium (Sodium hydroxide E524). After aseptic reconstitution with 10 ml of water for injections, each ml of concentrate contains 5 mg of decitabine (at pH 6.7 to 7.3). Within 15 minutes of reconstitution, the solution must be further diluted with cold infusion fluids (sodium chloride 9 mg/ml [0.9%] solution for injection or 5% glucose solution for injection) to a final concentration of 0.15 to 1.0 mg/ml.

WBC Count

WBC count can be determined, by any suitable institutional standard protocol. By way of example, suitable methods are described in Blumenreich [Reference 12] or Shafer [Reference 13], the contents of which are incorporated by reference.

SWOG Classification

SWOG classification was determined according to the criteria set out in Table 6 (Slovak et al, Reference 1; the contents of which are incorporated by reference). Cytogenetic testing can be carried out on peripheral blood, for example, in accordance with the standard methodology set forth in Slovak et al [Reference 1] or on bone marrow. Karyotyping or routine cytogenetic analysis involves the examination of chromosomes to identify structural abnormalities. Conventional cytogenetics (karyotyping) is the standard cytogenetic analysis for identifying gross chromosomal abnormalities: aneuploidies & structural (gain/loss, rearrangement). Chromosomes of a dividing human cell can be analysed clearly in white blood cells, specifically T lymphocytes, which are easily collected from blood. Cells from other tissues such as bone marrow, amniotic fluid, and other tissues can also be cultured for cytogenetic analysis. Cells from bone marrow are cultured for several days. Chromosomes in the growing and dividing cells are then fixed, spread on microscope slides, and stained to allow each of the chromosomes to be individually identified. Cytogenetic study was performed using the standard G-banding method (Reference 22, the contents of which are incorporated by reference). The distinct bands of each chromosome revealed by staining allow for analysis of the chromosomal structure (see Reference 14; the contents of which are incorporated by reference). Findings based on the FISH technique were used as considered supportive information (FISH analysis is based on probes directed to specific chromosomal regions (molecular cytogenetics)) (Reference 22).

Clinical Study

Methods: Eligible patients must be 70 years with AML previously untreated for whom the treatment of choice by physician assessment is low-intensity therapy, or the patient has refused standard induction chemotherapy; patients who received hypomethylating agents for prior MDS or MPD are excluded.

Results: 482 patients were treated with alternating cycles of decitabine and sapacitabine [decitabine 20 mg/m² infused over 1 hour intravenously for 5 consecutive days of a 4 week cycle (odd cycles), alternating with sapacitabine 300 mg orally b.i.d. for 3 days a week for 2 weeks of a 4 week cycle (even cycles)]. For 482 patients randomized to receive decitabine/sapacitabine (n=241) vs. decitabine only (n=241), randomization was stratified by the presence of antecedent MDS or MPN, peripheral white blood cell count (WBC <10,000 vs. ≥10,000) and bone marrow blast percentage (≥50% vs. <50%). Median age was 77 years (range 70-90), and 317 patients had de novo AML (66%), 165 secondary AML (34%). WBC was ≥10,000 in 161 patients (33%) and >40,000 in 59 patients (12%); 194 patients (40%) had unfavorable cytogenetic risk by SWOG criteria. Disease characteristics were well balanced in both treatment arms.

In total, 13.7% of patients achieved CR, more on the decitabine/sapacitabine vs. decitabine treatment (16.6% vs. 10.8%). A total of 37.3% treated patients received ≥5 cycles of treatment, similar for both treatments, as were 30- and 60-day death rates. Median overall survival was 5.9 months on the decitabine/sapacitabine treatment vs. 5.7 months on the decitabine treatment, which did not reach a statistically significant difference.

Table 1 shows the clinical outcomes of patients with <10,000 WBC (n=321), treated with either sapacitabine/decitabine (n=157) or decitabine only (n=162) and patients with ≥10,000 WBC treated with either sapacitabine/decitabine (n=84) or decitabine only (n=79). Median overall survival was higher on the sapacitabine/decitabine treatment vs. decitabine treatment (8.0 months vs. 5.8 months), as was CR rate (21.0% vs. 8.6%). Median CR duration was higher on the sapacitabine/decitabine treatment vs decitabine treatment (12.9 months vs. 10.4 months). Median overall survival was also higher for the patients treated with sapacitabine/decitabine with <10,000 WBC vs those with ≥10,000 WBC (8.0 months vs 3.8 months), as was CR rate (21.0% vs 8.3%). Median CR duration was also higher for the patients treated with sapacitabine/decitabine with <10,000 WBC vs those with treatment vs decitabine treatment (12.9 months vs. 10.4 months) with ≥10,000 WBC (12.9 months vs 4.7 months). In addition, for the patients treated with sapacitabine/decitabine with WBC <10,000 WBC vs those with ≥10,000 WBC 1-year survival was greater (42.0% vs 17.9%). For the subset of patients aged 80 years and over with <10,000 WBC the median overall survival was higher on the sapacitabine/decitabine treatment vs. decitabine treatment (5.9 months vs. 4.2 months).

Table 2 shows the clinical outcomes of patients with different SWOG classifications after either sapacitabine/decitabine or decitabine only treatment. For patients with SWOG category not unfavourable, median overall survival was higher on the sapacitabine/decitabine treatment vs. decitabine treatment (8.2 months vs. 5.7 months), as was CR rate (19.9% vs. 11.6%). Median overall survival was also higher for the SWOG non-unfavorable patients treated with sapacitabine/decitabine vs the SWOG unfavourable patients treated with sapacitabine/decitabine (8.2 months vs 3.8 months), as was CR rate (19.9% vs 12.0%). In addition, for the patients treated with sapacitabine/decitabine with SWOG not unfavourable vs those with SWOG unfavourable 1-year survival was greater (40.4% vs 24.0%).

Table 3 shows the clinical outcomes of patients with different SWOG classifications and different WBC counts after either sapacitabine/decitabine or decitabine only treatment. For patients with SWOG category not unfavourable and <10,000 WBC, median overall survival was higher on the sapacitabine/decitabine treatment vs. decitabine treatment (13.0 months vs. 5.6 months), as was CR rate (25.6% vs. 5.7%). Median overall survival was also higher for the patients with SWOG not unfavorable and <10,000 WBC vs those with SWOG unfavourable and ≥10,000 WBC, both treated with sapacitabine/decitabine (13.0 months vs 2.9 months), as was CR rate (25.6% vs 3.2%). In addition, for the patients treated with sapacitabine/decitabine with SWOG non-unfavorable and <10,000 WBC vs those with SWOG unfavourable and ≥10,000 WBC 1-year survival was greater (51.1% vs 9.7%).

Table 4 shows the influence of antecedent MDS/MPN on the clinical outcomes of patients after either sapacitabine/decitabine or decitabine only treatment. For patients with antecedent MDS/MPN, median overall survival was higher on the sapacitabine/decitabine treatment vs. decitabine treatment (6.4 months vs. 5.0 months), as was CR rate (16.7% vs. 5.7%). Median overall survival was also higher for patients with antecedent MDS/MPN treated with sapacitabine/decitabine vs the patients without antecedent MDS/MPN treated with sapacitabine/decitabine (6.4 months vs 5.9 months), as was CR duration (9.5 months vs 8.5 months). In addition, for the patients treated with sapacitabine/decitabine with antecedent MDS/MPN vs those without antecedent MDS/MPN 1-year survival was greater (34.8% vs 33.1%).

Table 5 shows the clinical outcomes of patients with or without antecedent MDS/MPN and different WBC counts after either sapacitabine/decitabine or decitabine only treatment. For patients with antecedent MDS/MPN and <10,000 WBC, median overall survival was higher on the sapacitabine/decitabine treatment vs. decitabine treatment (6.8 months vs. 4.9 months), as was CR rate (19.4% vs. 2.5%). Median overall survival was also higher for the patients with antecedent MDS/MPN and <10,000 WBC vs those without antecedent MDS/MPN and ≥10,000 WBC, both treated with sapacitabine/decitabine (6.8 months vs 3.8 months), as was CR rate (19.4% vs 5.6%). In addition, for the patients treated with sapacitabine/decitabine with antecedent MDS/MPN and <10,000 WBC vs those without antecedent MDS/MPN and ≥10,000 WBC 1-year survival was greater (41.7% vs 11.1%).

Various modifications and variations of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in the relevant fields are intended to be covered by the present invention.

### REFERENCES

1. Slovak et al; Blood 15 Dec. 2000, Vol 96, No. 13, 4075-4083; “Karyotypic analysis predicts outcome of preremission and postremission therapy in adult acute myeloid leukemia: a Southwest Oncology Group/Eastern Cooperative Oncology Group study”.

2. Raimondi S C, Ravindranath Y, Chang M N, et al. Chromosomal abnormalities in 478 children with acute myeloid leukemia: clinical characteristics and treatment outcome in a cooperative pediatric oncology group study—POG 8821. Blood.1999; 94: 3707-3716.

3. Bloomfield C D, Lawrence D, Byrd J C, et al. Frequency of prolonged remission duration after high-dose cytarabine intensification in acute myeloid leukemia varies by cytogenetic subtype. Cancer Res.1998; 58: 4173-4179.

4. Grimwade D, Walker H, Oliver F, et al. The importance of diagnostic cytogenetics on outcome in AML: analysis of 1,612 patients entered into the MRC AML 10 trial. Blood. 1998; 92: 2322-2333.

5. Slovak M L, Traweek S T, Willman C L, et al. Trisomy 11: an association with stem/progenitor cell immunophenotype. Br J Haematol.1995; 90:266-273.

6. Dastugue N, Payen C, Lafage-Pochitaloff M, et al. Prognostic significance of karyotype in de novo adult acute myeloid leukemia. Leukemia. 1995; 9:1491-1498.

7. Behm F G, Raimondi S C, Frestedt J L, et al. Rearrangement of the MLL gene confers a poor prognosis in childhood acute lymphoblastic leukemia, regardless of presenting age. Blood. 1996; 87: 2870-2877.

8. Schoch C, Haase D, Haferlach T, et al. Fifty-one patients with acute myeloid leukemia and translocation t(8;21)(q22;q22): an additional deletion in 9q is an adverse prognostic factor. Leukemia. 1996; 10:1288-1295.

9. Alsabeh R, Brynes R K, Slovak M L, Arber D A. Acute myeloid leukemia with t(6;9)(p23;q34): association with myelodysplasia, basophilia, and initial CD34 negative immunophenotype. Am J Clin Pathol. 1997; 107:430-437.

10. Vardiman J W et al, Blood, 1 Oct. 2002, Vol. 100, No. 7, page 2292-2302; “The WHO Classification of myeloid neoplasms”.

11. Harris N L, et al, J Clin Oncol, 1999, December; 17(12): 3835-49; “WHO classification of neoplastic diseases of the hematopoietic and lymphoid tissues”; report of the Clinical Advisory Committee meeting—Airlie House, Va., November 1997.

12. Blumenreich M S; Chapter 153; “The White Blood Cell and Differential Count”; Walker H K, Hall W D, Hurst J W, editors. Clinical Methods: The History, Physical, and Laboratory Examinations. 3rd edition. Boston: Butterworths; 1990.

13. Hematology, Basic Principles and Practice, 2nd ed., R. Hoffman, E. J. Benz, Jr., S. J. Shattil, et al., eds. New York: Churchill Livingstone, 1995, ISBN 0-443-08914-0, 2369; Chapter 157; “Preparation and Interpretation of Peripheral Blood Smears”, page 2202-2209; Shafer J A.

14. APPENDIX I: GENETIC TESTING METHODOLOGIES in Understanding Genetics: A New York, Mid-Atlantic Guide for Patients and Health Professionals Genetic Alliance; The New York-Mid-Atlantic Consortium for Genetic and Newborn Screening Services. Washington (D.C.): Genetic Alliance; 2009 Jul. 8. ISBN-13: 978-0-9821622-1-7.

15. Tallman M S et al. Drug therapy for acute myeloid leukemia. Blood, 106 (4): 1154-1163, 2005.

16. Tallman M S. Acute myeloid leukemia; decided victories, disappointments, and détente: a historical perspective. American Society of Hematology. Education Program Book, 2008.

17. Melchert M. Managing acute myeloid leukemia in the elderly. Oncology, 20 (13): 1674-1682, 2006.

18. Rowe J M et al. A phase 3 study of three induction regimens and of priming with GM-CSF in older adults with acute myeloid leukemia: a trial of the Eastern Cooperative Oncology Group. Blood, 103 (2): 479-485, 2004.

19. Giles F et al. The hematopoietic cell transplantation comorbidity index score is predictive of early death and survival in patients over 60 years of age receiving induction chemotherapy for acute myeloid leukemia. British Journal of Hematology, 136: 624-627, 2007.

20. “Unfavorable, Complex, and Monosomal Karyotypes: The Most Challenging Forms of Acute Myeloid Leukemia”; Johnnie J. Orozco, Frederick R. Appelbaum; Oncology (Williston Park, N.Y.), Volume 26, Issue 8, p. 706-12; Aug. 3, 2012; Vol 26; Issue 8.

21. Appelbaum F R, Kopecky K J, Tallman M S, et al. “The clinical spectrum of adult acute myeloid leukaemia associated with core binding factor translocations”; Brit J Haematol. 2006; 135:165-73.

22. Speicher, M. R. and Carter, N. P. Nat. Rev. Genet. 2005 6:782-79.

23. Vardiman J W et al. The 2008 revision of the WHO classification of myeloid neoplasms and acute leukemia: rationale and important changes. Blood, 114 (5): 937-951, 2009.

24. Swerdlow S H, Campo E, Harris N L, et al., editors. WHO Classification of Tumours of Haematopoietic and Lymphoid Tissues, Lyon, France: IARC; 2008.

25. Bowen D., Culligan D., Jowitt S., et al. Guidelines for the diagnosis and therapy of adult myelodysplastic syndromes; Br J Haematol. 2003; 120:187-200.

26. Kouides P. A., Bennett J M. Morphology and classification of the myelodysplastic syndromes and their pathologic variants; Semin Hematol. 1996; 33:95-110.

27. Bain B. J. The bone marrow aspirate of healthy subjects; Br J Haematol. 1996; 94:206-209.

28. Jones A V, Kreil S, Zoi K, et al. Widespread occurrence of the JAK2 V617F mutation in chronic myeloproliferative disorders. Blood. 2005; 106:2162-2168.

29. Levine R L, Loriaux M, Huntly B J, et al. The JAK2V617F activating mutation occurs in chronic myelomonocytic leukemia and acute myeloid leukemia, but not in acute lymphoblastic leukemia or chronic lymphocytic leukemia. Blood. 2005; 106: 3377-3379.

30. Steensma D P, Dewald G W, Lasho T L, et al. The JAK2 V617F activating tyrosine kinase mutation is an infrequent event in both “atypical” myeloproliferative disorders and myelodysplastic syndromes. Blood. 2005; 106:1207-1209.

31. Loh M L, Vattikuti S, Schubbert S, et al. Mutations in PTPN11 implicate the SHP-2 phosphatase in leukemogenesis. Blood. 2004; 103:2325-2331.

32. Tartaglia M, Niemeyer C M, Fragale A, et al. Somatic mutations in PTPN11 in juvenile myelomonocytic leukemia, myelodysplastic syndromes and acute myeloid leukemia. Nat Genet. 2003; 34:148-150.

33. Willman C L. Molecular genetic features of myelodysplastic syndromes (MDS). Leukemia. 1998; 12(suppl 1):S2-S6.

34. Hirsch-Ginsberg C, LeMaistre AC, Kantarjian H, et al. RAS mutations are rare events in Philadelphia chromosome-negative/bcr gene rearrangement-negative chronic myelogenous leukemia, but are prevalent in chronic myelomonocytic leukemia. Blood. 1990; 76:1214-1219.

35. Padua R A, Carter G, Hughes D, et al. RAS mutations in myelodysplasia detected by amplification, oligonucleotide hybridization, and transformation. Leukemia. 1988; 2:503-510.

36. Sugimoto K, Hirano N, Toyoshima H, et al. Mutations of the p53 gene in myelodysplastic syndrome (MDS) and MDS-derived leukemia. Blood. 1993; 81:3022-3026.

37. Tefferi A, Lasho T L, Gilliland G. JAK2 mutations in myeloproliferative disorders [letter]. N Engl J Med. 2005; 353:1416-1417, author reply 1416-1417.

38. Levine R L, Gilliland D G. Myeloproliferative disorders. Blood. 2008; 112:2190-2198.

39. Baxter E J, Scott L M, Campbell P J, et al. Acquired mutation of the tyrosine kinase JAK2 in human myeloproliferative disorders. Lancet. 2005; 365: 1054-1061.

40. Scott L M, Tong W, Levine R L, et al. JAK2 exon 12 mutations in polycythemia vera and idiopathic erythrocytosis. N Engl J Med. 2007; 356:459-468.

41. Pardanani A D, Levine R L, Lasho T, et al. MPL515 mutations in myeloproliferative and other myeloid disorders: a study of 1182 patients. Blood. 2006; 108:3472-3476.

TABLE 1
Impact of WBC count on outcomes of sapacitabine/decitabine and
decitabine only treatment

| | | Sapacitabine/ | |
|---|---|---|---|
| Parameter | WBC count | Decitabine | Decitabine |
| Median overall | <10,000 WBC | 8.0 | 5.8 |
| survival, months | | N = 157 | N = 162 |
| | ≥10,000 WBC | 3.8 | 5.5 |
| | | N = 84 | N = 79 |
| CR rate (%) | <10,000 WBC | 21.0 | 8.6 |
| | ≥10,000 WBC | 8.3 | 15.2 |
| CR median duration, | <10,000 WBC | 12.9 | 10.4 |
| months | ≥10,000 WBC | 4.7 | 10.1 |
| 1-year survival (%) | <10,000 WBC | 42.0 | 32.9 |
| | ≥10,000 WBC | 17.9 | 38.5 |
| Patients aged 80 and | <10,000 WBC | 5.9 | 4.2 |
| over, median overall | | | |
| survival, months | | | |

TABLE 2
Influence of SWOG cytogenetic classification on outcomes of
sapacitabine/decitabine and decitabine only treatment

| | SWOG | Sapacitabine/ | |
|---|---|---|---|
| Parameter | category | Decitabine | Decitabine |
| Median overall | Unfavourable | 3.8 | 5.7 |
| survival months | | N = 100 | N = 94 |
| | Not | 8.2 | 5.7 |
| | Unfavourable | N = 141 | N = 147 |
| CR rate (%) | Unfavourable | 12.0 | 9.6 |
| | Not | 19.9 | 11.6 |
| | Unfavourable | | |
| 1-year survival (%) | Unfavourable | 24.0 | 33.0 |
| | Not | 40.4 | 35.9 |
| | Unfavourable | | |

TABLE 3
Patient outcomes analysed by SWOG category and WBC count

| | | | Sapacitabine/ | |
|---|---|---|---|---|
| Parameter | SWOG | WBC | Decitabine | Decitabine |
| Median overall | Not Unfavourable | <10,000 | 13.0 | 5.6 |
| survival | | | N = 90 | N = 88 |
| months | Unfavourable | ≥10,000 | 2.9 | 3.0 |
| | | | N = 31 | N = 20 |
| CR rate (%) | Not unfavourable | <10,000 | 25.6 | 5.7 |
| | Unfavourable | ≥10,000 | 3.2 | 0 |
| 1-year survival | Not unfavourable | <10,000 | 51.1 | 31.8 |
| (%) | Unfavourable | ≥10,000 | 9.7 | 30.0 |

TABLE 4
Influence of antecedent MDS/MPN on outcomes of
sapacitabine/decitabine and decitabine only treatment

| | Antecedent | Sapacitabine/ | |
|---|---|---|---|
| Parameter | MDS/MPN | Decitabine | Decitabine |
| Median overall | Yes | 6.4 | 5.0 |
| survival, months | | N = 66 | N = 70 |
| | No | 5.9 | 6.7 |
| | | N = 175 | N = 171 |
| CR rate (%) | Yes | 16.7 | 5.7 |
| | No | 16.6 | 12.9 |
| CR median duration, | Yes | 9.5 | 7.1 |
| months | No | 8.5 | 10.4 |
| 1-year survival (%) | Yes | 34.8 | 32.9 |
| | No | 33.1 | 35.5 |

TABLE 5
Patient outcomes analysed by Antecedent MDS/MPN and WBC count

| | Antecedent | | Sapacitabine/ | |
|---|---|---|---|---|
| Parameter | MDS/MPN | WBC | Decitabine | Decitabine |
| Median overall | Yes | <10,000 | 6.8 | 4.9 |
| survival months | | | N = 36 | N = 40 |
| | No | ≥10,000 | 3.8 | 5.5 |
| | | | N = 54 | N = 49 |
| CR rate (%) | Yes | <10,000 | 19.4 | 2.5 |
| | No | ≥10,000 | 5.6 | 18.4 |
| 1-year survival (%) | Yes | <10,000 | 41.7 | 27.5 |
| | No | ≥10,000 | 11.1 | 36.7 |

TABLE 6
Southwestern Oncology Group and Medical Research Council cytogenetic risk
category definitions (source: Slovak et al; Blood 15 Dec. 2000, Vol 96, No. 13, 4075-4083)

| | | No. of patients | | No of patients |
|---|---|---|---|---|
| Risk status | SWOG coding. | (n = 609) | MRC coding | (n = 609) |
| Favorable | inv(16)/t(16;16)del(16q). t(15;17) with/without secondary | 121 (20%) | inv(16)/t(16;16)del(16q). t(15;17), t(6;21) | 130 (21%) |
| | aberrations: t(8;21) lacking del(9q) or complex karyotypes | | with/without secondary abn | |
| Intermediate | Normal, +8, +6, −Y, del(12p) | 278 (48%)* | Normal, 11q23 abn, +8 del(9q), del (7q), | 375 (62%) |
| | | | +21, +22 , all others | |
| Unfavorable | del(5q)/−5, −7/del(7q), abn 3q, 9q, 11q, 20q, 21q, 17p, | 184 (30%) | del(5q)/−6, −7, abn (3q), complex | 104 (17%) |
| | t(8;9), t(9;22) and complex karyotypes | | karyotypes (≥5 unreltaed abn) | |
| | (≥3 unrelated abn) | | t(9;22) and t(6;9)† | |
| Unknown | All other abnormalities | 26 (4%) | Category not recognized | — |

SWOG indicates Southwest Oncology Group;
MRC, Medical Research Council (United Kingdom);
abn, abnormality
*The intermediate group contains 244 patiens with normal karyotypes
†Risk status for t(6;9)or t(9;22) is not defined by MRC criteria, presumably due to a lack of these low frequency aberrations in their cohort.

TABLE 7
WHO classification of myeloid neoplasms and
myeloid dysplastic syndrome.
Major subgroups and the specific entities of which they are composed.

Myeloproliferative neoplasms (MPN)
Chronic myelogenous leukemia, BCR-ABL1-positive
Chronic neutrophilic leukemia
Polycythennia vera
Primary myelofibrosis
Essential thronnbocythennia
Chronic eosinophilic leukemia, not otherwise specified
Mastocytosis
Myeloproliferative neoplasms, unclassifiable
Myelodysplastic syndrome (MDS)
Refractory cytopenia with unilineage dysplasia
Refractory anemia
Refractory neutropenia
Refractory thrombocytopenia
Refractory anemia with ring sideroblasts
Refractory cytopenia with multilineage dysplasia
Refractory anemia with excess blasts
Myelodysplastic syndrome with isolated del(5q)
Myelodysplastic syndrome, unclassifiable
Childhood myelodysplastic syndrome
Provisional entity: refractory cytopenia of childhood
Myelodysplastic/myeloproliferative neoplasms (MDS/MPN)
Chronic myelomonocytic leukemia
Atypical chronic myeloid leukemia, BCR-ABL1-negative
Juvenile myelomonocytic leukemia
Myelodysplastic/myeloproliferative neoplasm, unclassifiable
Provisional entity: refractory anemia with ring sideroblasts and
thrombocytosis

TABLE 8
Recurring chromosomal abnormalities considered as presumptive evidence
of MDS in the setting of persistent cytopenia of undetermined origin,
but in the absence of definitive morphologic features of MDS

| Unbalanced abnormalities | Balanced abnormalities |
|---|---|
| −7 or del(7q) | t(11;16)(q23;p13.3) |
| −5 or del(5q) | t(3;21)(q26.2;q22.1) |
| i(17q) or t(17p) | t(1;3)(p36.3;q21.1) |
| −13 or del(13q) | t(2;11)(p21;q23) |
| del(11q) | inv(3)(q21q26.2) |
| del(12p) or t(12p) | t(6;9)(p23;q34) |
| del(9q) | |
| idic(X)(q13) | |

Complex karyotype (3 or more chromosomal abnormalities) involving one or more of the above abnormalities.

TABLE 9
Criteria for polycythemia vera (PV)
Diagnosis requires the presence of both major criteria and
one minor criterion or the presence of the first major criterion
together with two minor criteria:

Major criteria
1. Hemoglobin > 18.5 g/dL in men, 16.5 g/dL in women or other
evidence of increased red cell volume*
2. Presence of JAK2 V617F or other functionally similar mutation such
as JAK2 exon 12 mutation
Minor criteria
1. Bone marrow biopsy showing hypercellularity for age with trilineage
growth (panmyelosis) with prominent erythroid, granulocytic, and
megakaryocytic proliferation
2. Serum erythropoietin level below the reference range for normal
3. Endogenous erythroid colony formation in vitro
*Hemoglobin or hematocrit > 99th percentile of method-specific reference range for age, sex, altitude of residence or hemoglobin > 17 g/dL in men, 15 g/dL in women if associated with a documented and sustained increase of at least 2 g/dLfrom a person's baseline value that cannot be attributed to correction of iron deficiency or elevated red cell mass > 25% above mean normal predicted value.

TABLE 10
Criteria for essential thrombocythemia (ET)
Diagnosis requires meeting all 4 criteria

1. Sustained platelet count ≥ 450 × 10⁹/L*
2. Bone marrow biopsy specimen showing proliferation mainly of the
megakaryocytic lineage with increased numbers of enlarged, mature
megakaryocytes. No significant increase or left-shift of neutrophil
granulopoiesis or erythropoiesis.
3. Not meeting WHO criteria for polycythemia vera,† primary
myelofibrosis,‡ BCR-ABL1-positive CML,§ or myelodysplastic
syndrome,|| or other myeloid neoplasm.
4. Demonstration of JAK2 V617F or other clonal marker, or in the
absence of JAK2 V617F, no evidence of reactive thrombocytosis¶.
*Sustained during the work-up process.
†Requires the failure of iron replacement therapy to increase hemoglobin level to the polycythemia vera range in the presence of decreased serum ferritin. Exclusion of polycythemia vera is based on hemoglobin and hematocrit levels, and red cell mass measurement is not required.
‡Requires the absence of relevant reticulin fibrosis, collagen fibrosis, peripheral blood leukoerythroblastosis, or markedly hypercellular marrow accompanied by megakaryocyte morphology that is typical for primary myelofibrosis - small to large megakaryocytes with an aberrant nuclear/cytoplasmic ratio and hyperchromatic, bulbous, or irregularly folded nuclei and dense clustering.
§Requires the absence of BCR-ABL1.
||Requires the absence of dyserythropoiesis and dysgranulopoiesis.
¶Causes of reactive thrombocytosis include iron deficiency, splenectomy, surgery, infection, inflammation, connective tissue disease, metastatic cancer, and lymphoproliferative disorders. However, the presence of a condition associated with reactive thrombocytosis does not exclude the possibility of ET if other criteria are met.

TABLE 11
Criteria for primary myelofibrosis (PMF)
Diagnosis requires meeting all 3 major criteria and 2 minor criteria

Major criteria
1. Presence of megakaryocyte proliferation and atypia,* usually
accompanied by either reticulin or collagen fibrosis,
or,
in the absence of significant reticulin fibrosis, the megakaryocyte changes
must be accompanied by an increased bone marrow cellularity
characterized by granulocytic proliferation and often decreased
erythropoiesis (ie, prefibrotic cellular-phase disease)
2. Not meeting WHO criteria for polycythemia vera,† BCR-ABL1-
positive chronic myelogenous leukemia,‡ myelodysplastic
syndrome,§ or other myeloid disorders
3. Demonstration of JAK2 V617F or other clonal marker (eg,
MPLW515K/L),
or,
in the absence of the above clonal markers, no evidence that bone marrow
fibrosis is secondary to infection, autoimmune disorder or other chronic
inflammatory condition, hairy cell leukemia or other lymphoid neoplasm,
metastatic malignancy, or toxic (chronic) myelopathies||
Minor criteria
1. Leukoerythroblastosis¶
2. Increase in serum lactate dehydrogenase level¶
3. Anemia¶
4. Palpable splenomegaly¶
*Small to large megakaryocytes with an aberrant nuclear/cytoplasmic ratio and hyperchromatic, bulbous, or irregularly folded nuclei and dense clustering.
†Requires the failure of iron replacement therapy to increase hemoglobin level to the polycythemia vera range in the presence of decreased serum ferritin. Exclusion of polycythemia vera is based on hemoglobin and hematocrit levels. Red cell mass measurement is not required.
‡Requires the absence of BCR-ABL1.
§Requires the absence of dyserythropoiesis and dysgranulopoiesis.
||It should be noted that patients with conditions associated with reactive myelofibrosis are not immune to primary myelofibrosis, and the diagnosis should be considered in such cases if other criteria are met.
¶Degree of abnormality could be borderline or marked.

1. A method of treating AML in a subject, wherein said subject:
(I) has a white blood cell (WBC) count of less than about 10,000 cells/microliter, and/or
(II) has a cytogenetic risk classification according to the US Southwest Oncology Group (SWOG) that is not unfavourable, and/or
(III) falls within a classification selected from antecedent myelodysplastic syndrome (MDS), antecedent myeloproliferative neoplasm (MPN), and antecedent myelodysplastic/myeloproliferative neoplasm (MDS/MPN);
said method comprising administering to the subject a therapeutically effective amount of (i) sapacitabine, or a metabolite thereof; and (ii) decitabine; in accordance with a dosing regimen comprising at least one first treatment cycle and at least one second treatment cycle,
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

2. A method according to claim 1 wherein the second treatment cycle comprises administering a therapeutically effective amount of sapacitabine.

3. A method according to claim 1 wherein the metabolite of sapacitabine is CNDAC.

4. A method according to claim 1 wherein said first treatment cycle comprises administering a therapeutically effective amount of decitabine for 5 consecutive days followed by a rest period of from 3 to 5 weeks, or until treatment-related toxicities are resolved, whichever is longer.

5. A method according to claim 1 wherein said first treatment cycle comprises administering a therapeutically effective amount of decitabine for 5 consecutive days followed by a 3 week rest period.

6. A method according to claim 1 wherein said second treatment cycle comprises administering a therapeutically effective amount of sapacitabine or metabolite thereof for 3 consecutive days per week, for 2 weeks followed by a 2 week rest period

7. A method according to claim 1 which comprises two or more of each treatment cycle.

8. A method according to claim 1 which comprises two to four of each treatment cycle.

9. A method according to claim 1 wherein the decitabine is administered intravenously.

10. A method according to claim 1 wherein the decitabine is administered in a dose of from about 10 to about 20 mg/m².

11. A method according to claim 1 wherein the decitabine is administered in a dose of about 20 mg/m² per day.

12. A method according to claim 1 wherein the decitabine is administered by intravenous infusion over a period of about 1 hour.

13. A method according to claim 1 wherein the sapacitabine or metabolite thereof is administered orally.

14. A method according to claim 13 wherein the sapacitabine or metabolite thereof is administered in a dose of about 100-400 mg b.i.d., more preferably from about 250-300 mg b.i.d.

15. A method according to claim 14 wherein the sapacitabine or metabolite thereof is administered in a dose of about 300 mg b.i.d.

16. A method according to claim 1 wherein the subject is an elderly subject.

17. A method according to claim 16 wherein the subject is 70 years of age or over.

18. A method of treating AML in an elderly subject, wherein said subject:
(I) has a white blood cell (WBC) count of less than about 10,000 cells/microliter and/or
(II) has a cytogenetic risk classification according to the US Southwest Oncology Group (SWOG) that is not unfavourable, and/or
(III) falls within a classification selected from antecedent myelodysplastic syndrome (MDS), antecedent myeloproliferative neoplasm (MPN), and antecedent myelodysplastic/myeloproliferative neoplasm (MDS/MPN);
said method comprising administering to a subject a therapeutically effective amount of (i) sapacitabine; and (ii) decitabine; in accordance with a dosing regimen comprising at least one first treatment cycle and at least one second treatment cycle,
wherein said first treatment cycle comprises administering decitabine intravenously in a dose of about 20 mg/m²per day for 5 to 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

19-28. (canceled)

29. A kit of parts comprising:
(i) sapacitabine, or a metabolite thereof;
(ii) decitabine; and
(iii) instructions for administering sapacitabine, or a metabolite thereof, and decitabine to a subject, in accordance with a dosing regimen comprising at least one first treatment cycle and at least one second treatment cycle, wherein said subject:
(I) has a white blood cell (WBC) count of less than about 10,000 cells/microliter and/or
(II) has a cytogenetic risk classification according to the US Southwest Oncology Group (SWOG) that is not unfavourable, and/or
(III) falls within a classification selected from antecedent myelodysplastic syndrome (MDS), antecedent myeloproliferative neoplasm (MPN), and antecedent myelodysplastic/myeloproliferative neoplasm (MDS/MPN);
wherein said first treatment cycle comprises administering a therapeutically effective amount of decitabine for 5 to 10 consecutive days followed by a rest period of from 3 to 5 weeks, or until treatment-related toxicities are resolved, whichever is longer; and
wherein said second treatment cycle comprises administering a therapeutically effective amount of sapacitabine, or a metabolite thereof, for 3 consecutive days per week, for 2 weeks followed by a rest period of from 2 to 4 weeks, or until treatment-related toxicities are resolved, whichever is longer.

30. The kit of parts according to claim 29,
wherein said first treatment cycle comprises administering decitabine intravenously in a dose of about 20 mg/m² for 5 to 10 consecutive days followed by a 3 to 5 week rest period, or until treatment-related toxicities are resolved, whichever is longer; and
wherein said second treatment cycle comprises administering sapacitabine orally in a dose of about 300 mg b.i.d. for 3 consecutive days per week, for 2 weeks followed by a 2 to 4 week rest period, or until treatment-related toxicities are resolved, whichever is longer.

31-32. (canceled)
